(19) United States
(12) Patent Application Publication
(10) Pub. No.: US 2020/0316083 A1
(43) Pub. Date: Oct. 8, 2020

(54) ANTIBACTERIAL COMPOSITIONS

(71) Applicant: WOCKHARDT LIMITED, Chilkalthana, Aurangabad, (IN)

(72) Inventors: Sachin Subhash BHAGWAT, Maharashtra Aurangabad 5 (IN); Mahesh Vitthalbhai PATEL, Maharashtra Aurangabad 3, (IN); Vikas Vithalrao DESHMUKH, Maharashtra, Ahmednagar (IN)

(73) Assignee: WOCKHARDT LIMITED, Chikalthana, Aurangabad (IN)

(21) Appl. No.: 16/305,361

(22) PCT Filed: Jun. 16, 2017
(86) PCT No.: PCT/IB2017/053587

§ 371 Date: Nov. 28, 2018

(30) Foreign Application Priority Data
Jun. 17, 2016 (IN) 201621020848
Jun. 17, 2016 (IN) 201621020849

(51) Int. Cl. A61K 31/551; A61K 31/547; A61K 31/431; A61K 31/424; A61P 31/04

(52) U.S. Cl. CPC A61K 31/551; A61K 31/547; A61P 31/04; A61K 31/424; A61K 31/431

(57) ABSTRACT

Pharmaceutical composition comprising beta-lactamase inhibitors or a pharmaceutically acceptable salt, and a compound of Formula (I) or a pharmaceutically acceptable salt thereof are disclosed.

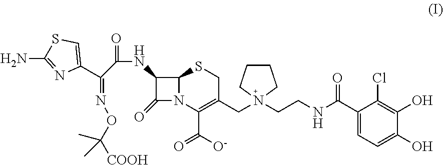

### RELATED PATENT APPLICATIONS

This application claims priority to and benefit of the Indian Patent Application Nos. 201621020848 (filed Jun. 17, 2016) and 201621020849 (filed Jun. 17, 2016), the disclosures of which are incorporated herein by reference in its entirety as if fully rewritten herein.

### FIELD OF THE INVENTION

The invention relates to antibacterial compositions and methods for treatment, control or prevention of bacterial infections.

### BACKGROUND OF THE INVENTION

Bacterial infections continue to remain one of the major causes contributing towards human diseases. One of the key challenges in treatment of bacterial infections is the ability of bacteria to develop resistance to one or more antibacterial agents over time. Examples of such bacteria that have developed resistance to typical antibacterial agents include: Penicillin-resistant Streptococcus pneumoniae, Vancomycin-resistant Enterococci, and Methicillin-resistant Staphylococcus aureus. The problem of emerging drug-resistance in bacteria is often tackled by switching to newer antibacterial agents, which can be more expensive and sometimes more toxic. Additionally, this may not be a permanent solution as the bacteria often develop resistance to the newer antibacterial agents as well in due course. In general, bacteria are particularly efficient in developing resistance, because of their ability to multiply very rapidly and pass on the resistance genes as they replicate. Therefore, there is a need for development of newer ways to treat infections that are becoming resistant to known therapies and methods.

Surprisingly, it has been found that the compositions comprising a compound of Formula (I) or a pharmaceutically acceptable salt thereof and at least one beta-lactamase inhibitor or a pharmaceutically acceptable salt thereof, exhibit synergistic antibacterial activity, even against resistant bacterial strains.

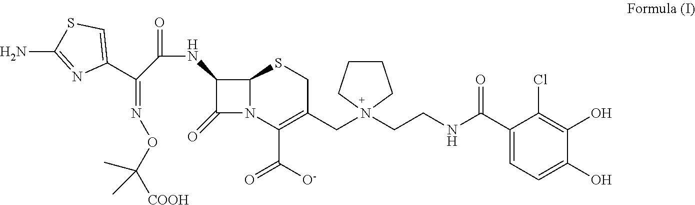

### SUMMARY OF THE INVENTION

Accordingly, there are provided pharmaceutical compositions comprising: (a) at least one beta-lactamase inhibitor, or a pharmaceutically acceptable salt thereof; and (b) a compound of Formula (I):

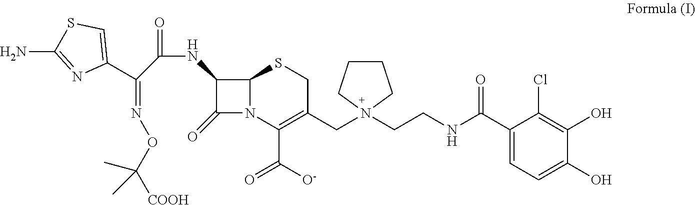

or a pharmaceutically acceptable salt thereof.

In another general aspect, there are provided methods for treating or preventing a bacterial infection in a subject, said methods comprising administering to said subject an effective amount of a pharmaceutical composition comprising: (a) at least one beta-lactamase inhibitor, or a pharmaceutically acceptable salt thereof; and (b) a compound of Formula (I), or a pharmaceutically acceptable salt thereof.

In another general aspect, there are provided methods for treating or preventing a bacterial infection in a subject, said methods comprising administering to said subject an effective amount of: (a) at least one beta-lactamase inhibitor, or a pharmaceutically acceptable salt thereof; and (b) a compound of Formula (I), or a pharmaceutically acceptable salt thereof.

The details of one or more embodiments of the invention are set forth in the description below. Other features, objects and advantages of the invention will be apparent from the following description including claims.

### DETAILED DESCRIPTION OF THE INVENTION

Reference will now be made to the exemplary embodiments, and specific language will be used herein to describe the same. It should nevertheless be understood that no limitation of the scope of the invention is thereby intended. Alterations and further modifications of the inventive features illustrated herein, which would occur to one skilled in the relevant art and having possession of this disclosure, are to be considered within the scope of the invention. It must be noted that, as used in this specification and the appended claims, the singular forms “a”, “an”, and “the” include plural referents unless the content clearly dictates otherwise. All references including patents, patent applications, and literature cited in the specification are expressly incorporated herein by reference in their entirety as if fully rewritten herein.

The inventors have discovered that a pharmaceutical composition comprising: (a) at least one beta-lactamase inhibitor, or a pharmaceutically acceptable salt thereof; and (b) a compound of Formula (I):

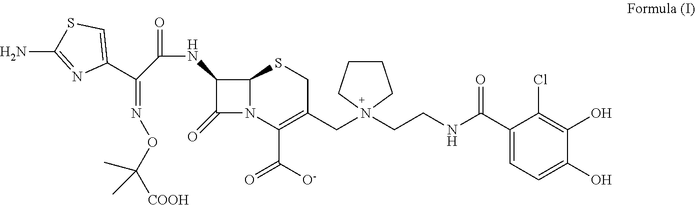

or a pharmaceutically acceptable salt thereof, exhibits unexpectedly improved antibacterial efficacy, even against resistant bacteria, including those producing extended spectrum beta-lactamase enzymes (ESBLs).

The term “infection” or “bacterial infection” as used herein includes presence of bacteria, in or on a subject, which, if its growth were inhibited, would result in a benefit to the subject. As such, the term “infection” in addition to referring to the presence of bacteria also refers to presence of other floras, which are not desirable. The term “infection” includes infection caused by bacteria.

The term “treat”, “treating” or “treatment” as used herein refers to administration of a medicament, including a pharmaceutical composition, or one or more pharmaceutically active ingredients, for prophylactic and/or therapeutic purposes. The term “prophylactic treatment” refers to treating a subject who is not yet infected, but who is susceptible to, or otherwise at a risk of infection (preventing the bacterial infection). The term “therapeutic treatment” refers to administering treatment to a subject already suffering from infection. The terms “treat”, “treating” or “treatment” as used herein also refer to administering compositions, or one or more of pharmaceutically active ingredients discussed herein, with or without additional pharmaceutically active or inert ingredients, in order to: (i) reduce or eliminate either a bacterial infection, or one or more symptoms of a bacterial infection, or (ii) retard progression of a bacterial infection, or one or more symptoms of a bacterial infection, or (iii) reduce severity of a bacterial infection, or one or more symptoms of a bacterial infection, or (iv) suppress clinical manifestation of a bacterial infection, or (v) suppress manifestation of adverse symptoms of a bacterial infection.

The terms “pharmaceutically effective amount” or “therapeutically effective amount” or “effective amount” as used herein refer to an amount, which has a therapeutic effect or is the amount required to produce a therapeutic effect in a subject. For example, a “therapeutically effective amount” or “pharmaceutically effective amount” or “effective amount” of an antibacterial agent or a pharmaceutical composition is the amount of the antibacterial agent or the pharmaceutical composition required to produce a desired therapeutic effect as may be judged by clinical trial results, model animal infection studies, and/or in vitro studies (e.g. in agar or broth media). Such effective amount depends on several factors, including but not limited to, the microorganism (e.g. bacteria) involved, characteristics of the subject (for example height, weight, sex, age and medical history), severity of infection and particular type of the antibacterial agent used. For prophylactic treatments, a prophylactically effective amount is that amount which would be effective in preventing the bacterial infection.

The term “administration” or “administering” refers to and includes delivery of a composition, or one or more pharmaceutically active ingredients to a subject, including for example, by any appropriate method, which serves to deliver the composition or its active ingredients or other pharmaceutically active ingredients to the site of infection. The method of administration may vary depending on various factors, such as for example, the components of the pharmaceutical composition or type/nature of the pharmaceutically active or inert ingredients, site of the potential or actual infection, the microorganism involved, severity of the infection, age and physical condition of the subject and a like. Some non-limiting examples of ways to administer a composition or a pharmaceutically active ingredient to a subject according to this invention include oral, intravenous, topical, intrarespiratory, intraperitoneal, intramuscular, parenteral, sublingual, transdermal, intranasal, aerosol, intraocular, intratracheal, intrarectal, vaginal, gene gun, dermal patch, eye drop and mouthwash. In case of a pharmaceutical composition comprising more than one ingredients (active or inert), one of the ways of administering such composition is by admixing the ingredients (e.g. in the form of a suitable unit dosage form such as tablet, capsule, solution, powder or a like) and then administering the dosage form. Alternatively, the ingredients may also be administered separately (simultaneously or one after the other) as long as these ingredients reach beneficial therapeutic levels such that the composition as a whole provides a synergistic and/or desired effect.

The term “growth” as used herein refers to a growth of one or more microorganisms and includes reproduction or population expansion of the microorganism (e.g. bacteria). The term “growth” also includes maintenance of on-going metabolic processes of the microorganism, including the processes that keep the microorganism alive.

The term, “effectiveness” as used herein refers to ability of a treatment, or a composition, or one or more pharmaceutically active ingredients to produce a desired biological effect in a subject. For example, the term “antibacterial effectiveness” of a composition or of an antibacterial agent refers to the ability of the composition or the antibacterial agent to prevent or treat bacterial infection in a subject.

The term “synergistic” or “synergy” as used herein refers to the interaction of two or more agents so that their combined effect is greater than their individual effects.

The term “antibacterial agent” as used herein refers to any substance, compound, a combination of substances, or a combination of compounds capable of: (i) inhibiting, reducing or preventing growth of bacteria; (ii) inhibiting or reducing ability of a bacteria to produce infection in a subject; or (iii) inhibiting or reducing ability of bacteria to multiply or remain infective in the environment. The term “antibacterial agent” also refers to compounds capable of decreasing infectivity or virulence of bacteria.

The term “beta-lactam antibacterial agent” as used herein refers to compounds with antibacterial properties and containing a beta-lactam nucleus in their molecular structure.

The term “beta-lactamase” or “beta-lactamase enzyme” as used herein refers to any enzyme or protein or any other substance that breaks down a beta-lactam ring. The term “beta-lactamase” includes enzymes that are produced by bacteria and have the ability to hydrolyse the beta-lactam ring in a beta-lactam compound, either partially or completely.

The term “extended spectrum beta-lactamase” (ESBL) as used herein includes those beta-lactamase enzymes, which are capable of conferring bacterial resistance to various beta-lactam antibacterial agents such as penicillins, cephalosporins, aztreonam and the like.

The term “beta-lactamase inhibitor” as used herein refers to a compound capable of inhibiting activity of one or more beta-lactamase enzymes, either partially or completely.

The term “colony forming units” or “CFU” as used herein refers to an estimate of number of viable bacterial cells per ml of the sample. Typically, a “colony of bacteria” refers to a mass of individual bacteria growing together.

The term “pharmaceutically inert ingredient” or “carrier” or “excipient” refers to and includes compounds or materials used to facilitate administration of a compound, for example, to increase the solubility of the compound. Typical, non-limiting examples of solid carriers include starch, lactose, dicalcium phosphate, sucrose, and kaolin. Typical, non-limiting examples of liquid carriers include sterile water, saline, buffers, non-ionic surfactants, and edible oils. In addition, various adjuvants commonly used in the art may also be included. These and other such compounds are described in literature, e.g., in the Merck Index (Merck & Company, Rahway, N.J.). Considerations for inclusion of various components in pharmaceutical compositions are described, e.g., in Gilman et al. (Goodman and Gilman's: The Pharmacological Basis of Therapeutics, 8th Ed., Pergamon Press., 1990), which is incorporated herein by reference in its entirety.

The term “subject” as used herein refers to vertebrate or invertebrate, including a mammal. The term “subject” includes human, animal, a bird, a fish, or an amphibian. Typical, non-limiting examples of a “subject” include humans, cats, dogs, horses, sheep, bovine cows, pigs, lambs, rats, mice and guinea pigs.

The term “pharmaceutically acceptable salt” as used herein refers to one or more salts of a given compound which possesses desired pharmacological activity of the free compound and which is neither biologically nor otherwise undesirable. In general, the term “pharmaceutically acceptable salts” refer to salts that are suitable for use in contact with the tissues of human and animals without undue toxicity, irritation, allergic response and the like, and are commensurate with a reasonable benefit/risk ratio. Pharmaceutically acceptable salts are well known in the art. For example, S. M. Berge, et al. (J. Pharmaceutical Sciences, 66; 1-19, 1977), incorporated herein by reference in its entirety, describes various pharmaceutically acceptable salts in details.

The term “stereoisomer” as used herein refers to and includes isomeric molecules that have the same molecular formula but differ in positioning of atoms and/or functional groups in the space. Stereoisomers may further be classified as enantiomers (where different isomers are mirror-images of each other) and diastereomers (where different isomers are not mirror-images of each other). Diastereomers include isomers such as conformers, meso compounds, cis-trans (E-Z) isomers, and non-enantiomeric optical isomers.

A person of skills in the art would appreciate that various compounds described herein (including, for example the compound of Formula (I) and the beta-lactamase inhibitor) can exist and are often used as their pharmaceutically acceptable derivatives (such as salts, pro-drugs, metabolites, esters, ethers, hydrates, polymorphs, solvates, complexes, and adducts).

In one general aspect, there are provided pharmaceutical compositions comprising: (a) at least one beta-lactamase inhibitor, or a pharmaceutically acceptable salt thereof; and (b) a compound of Formula (I):

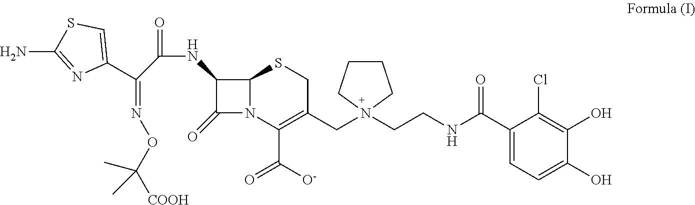

or a pharmaceutically acceptable salt thereof.

Chemically, the compound of Formula (I) may also be referred to as “Pyrrolidinium, 1-[[(6R,7R)-7-[[(2Z)-2-(2-amino-4-thiazolyl)-2-[(1-carboxy-1-methylethoxy)imino]acetyl]amino]-2-carboxy-8-oxo-5-thia-1-azabicyclo[4.2.0]oct-2-en-3-yl]methyl]-1-[2-[(2-chloro-3,4-dihydroxy-benzoyl)amino]ethyl]-, inner salt”; or “(6R,7R)-7-[(2Z)-2-(2-amino-1,3-thiazol-4-yl)-2-{[(2-carboxypropan-2-yl)oxy]imino}acetamido]-3-({1-[2-(2-chloro-3,4-dihydroxybenzamido)ethyl]pyrrolidin-1-ium-1-yl}methyl)-8-oxo-5-thia-1-azabicyclo[4.2.0]oct-2-ene-2-carboxylate].

A wide variety of beta-lactamase inhibitors can be used according to the invention. In some embodiments, the beta-lactamase inhibitor is at least one selected from the group consisting of sulbactam, tazobactam, clavulanic acid, and avibactam.

In some other embodiments, the beta-lactamase inhibitor is a compound of Formula (II):

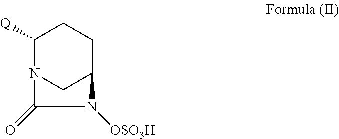

or a stereoisomer thereof,

wherein Q is:
(a) cyano;
(b) five to fourteen membered heteroaryl, optionally substituted with one or more of the following:
   (i) —CO—NH₂,
   (ii) five to fourteen membered heteroaryl,
   (iii) three to seven membered heterocycloalkyl,
   (iv) three to seven membered cycloalkyl,
   (v) five to fourteen membered aryl, or
   (vi) C₁-C₆ alkyl, optionally substituted with —NH₂ or three to seven membered heterocycloalkyl;
(c) —CO—NH—NH—CO—R₁;
(d) —CO—NH—O—R₁; or
(e) —CO—NH—R₁;

R₁ is
(a) hydrogen;
(b) three to seven membered heterocycloalkyl, optionally substituted with hydroxy group;
(c) C₁-C₆ alkyl, optionally substituted with: (i) three to seven membered heterocycloalkyl; (ii) one or more hydroxy groups; or (ii) one or more amino groups.

In some other embodiments, the beta-lactamase inhibitor is at least one compound selected from a group consisting of:

| Compound | Structure |
|---|---|
| Compound (B) | 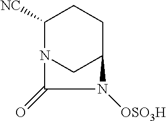 |
| Compound (C) | 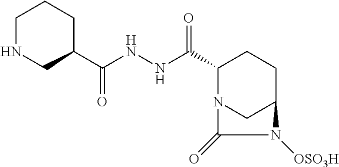 |
| Compound (D) | 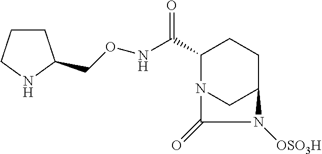 |
| Compound (E) | 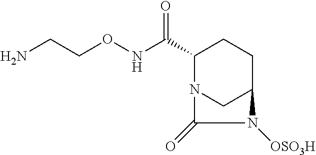 |
| Compound (F) | 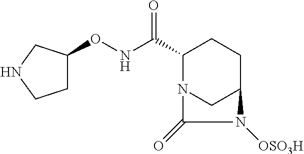 |
| Compound (G) | 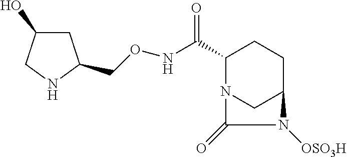 |
| Compound (H) | 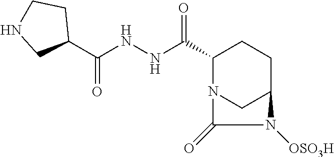 |
| Compound (J) | 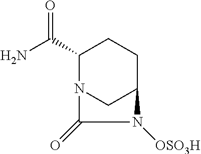 |
| Compound (K) | 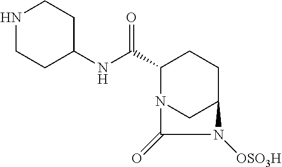 |
| Compound (L) | 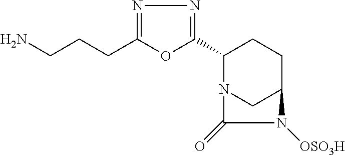 |

or a stereoisomer or a pharmaceutically acceptable salt thereof.

Both, the beta-lactamase inhibitor and the compound of Formula (I) may be present in the composition in their free forms or in the form of their pharmaceutically acceptable derivatives (such as salts, pro-drugs, metabolites, esters, ethers, hydrates, polymorphs, solvates, complexes, or adducts).

Individual amounts of the beta-lactamase inhibitor or a pharmaceutically acceptable salt thereof, and the compound of Formula (I) or a pharmaceutically acceptable salt thereof in the composition may vary depending on clinical requirements. In some embodiments, the beta-lactamase inhibitor or a pharmaceutically acceptable salt thereof is present in the composition in an amount from about 0.01 gram to about 10 gram. In some other embodiments, the compound of Formula (I) or a pharmaceutically acceptable salt thereof is present in the composition in an amount from about 0.01 gram to about 10 gram. In some other embodiments, the beta-lactamase inhibitor, or a pharmaceutically acceptable salt thereof is present in the composition in an amount from about 0.125 to about 4 gram per gram of the compound of Formula (I) or a pharmaceutically acceptable salt thereof.

In some embodiments, the pharmaceutical compositions according to the invention comprise about 1 gram of the compound of Formula (I) or a pharmaceutically acceptable salt thereof, and about 0.25 gram of the beta-lactamase inhibitor, or a pharmaceutically acceptable salt thereof;

In some embodiments, the pharmaceutical compositions according to the invention comprise about 1 gram of the compound of Formula (I) or a pharmaceutically acceptable salt thereof, and about 0.50 gram of the beta-lactamase inhibitor, or a pharmaceutically acceptable salt thereof;

In some embodiments, the pharmaceutical compositions according to the invention comprise about 1 gram of the compound of Formula (I) or a pharmaceutically acceptable salt thereof, and about 1 gram of the beta-lactamase inhibitor, or a pharmaceutically acceptable salt thereof;

In some embodiments, the pharmaceutical compositions according to the invention comprise about 1 gram of the compound of Formula (I) or a pharmaceutically acceptable salt thereof, and about 2 gram of the beta-lactamase inhibitor, or a pharmaceutically acceptable salt thereof;

In some embodiments, the pharmaceutical compositions according to the invention comprise about 2 gram of the compound of Formula (I) or a pharmaceutically acceptable salt thereof, and about 0.25 gram of the beta-lactamase inhibitor, or a pharmaceutically acceptable salt thereof;

In some embodiments, the pharmaceutical compositions according to the invention comprise about 2 gram of the compound of Formula (I) or a pharmaceutically acceptable salt thereof, and about 0.5 gram of the beta-lactamase inhibitor, or a pharmaceutically acceptable salt thereof;

In some embodiments, the pharmaceutical compositions according to the invention comprise about 2 gram of the compound of Formula (I) or a pharmaceutically acceptable salt thereof, and about 1 gram of the beta-lactamase inhibitor, or a pharmaceutically acceptable salt thereof;

In some embodiments, the pharmaceutical compositions according to the invention comprise about 2 gram of the compound of Formula (I) or a pharmaceutically acceptable salt thereof, and about 2 gram of the beta-lactamase inhibitor, or a pharmaceutically acceptable salt thereof;

In some embodiments, the pharmaceutical compositions according to the invention comprise about 0.5 gram of the compound of Formula (I) or a pharmaceutically acceptable salt thereof, and about 1 gram of the beta-lactamase inhibitor, or a pharmaceutically acceptable salt thereof; or

In some embodiments, the pharmaceutical compositions according to the invention comprise about 0.5 gram of the compound of Formula (I) or a pharmaceutically acceptable salt thereof, and about 2 gram of the beta-lactamase inhibitor, or a pharmaceutically acceptable salt thereof.

The pharmaceutical compositions according to the invention may include one or more pharmaceutically acceptable carriers or excipients or the like. Typical, non-limiting examples of such carriers or excipients include mannitol, lactose, starch, magnesium stearate, sodium saccharine, talcum, cellulose, sodium cro scarmello se, glucose, gelatine, sucrose, magnesium carbonate, wetting agents, emulsifying agents, solubilizing agents, buffering agents, lubricants, preservatives, stabilizing agents, binding agents and the like.

The pharmaceutical compositions or the active ingredients according to the present invention may be formulated into a variety of dosage forms, such as solid, semi-solid, liquid and aerosol dosage forms. Typical, non-limiting examples of some dosage forms include tablets, capsules, powders, solutions, suspensions, suppositories, aerosols, granules, emulsions, syrups, elixirs and the like.

In some embodiments, pharmaceutical compositions according to the invention are in the form of a powder or a solution. In some other embodiments, pharmaceutical compositions according to the invention are present in the form of a powder or a solution that can be reconstituted by addition of a compatible reconstitution diluent prior to administration. In some other embodiments, pharmaceutical compositions according to the invention are in the form of a frozen composition that can be diluted with a compatible reconstitution diluent prior to administration. Typical, non-limiting example of suitable compatible reconstitution diluent includes water.

In some other embodiments, pharmaceutical compositions according to the invention are present in the form ready to use for parenteral administration.

The compositions according to the invention can be formulated into various dosage forms wherein the active ingredients and/or excipients may be present either together (e.g. as an admixture) or as separate components. When the various ingredients in the composition are formulated as a mixture, such compositions can be delivered by administering such a mixture to a subject using any suitable route of administration. Alternatively, pharmaceutical compositions according to the invention may also be formulated into a dosage form wherein one or more ingredients (such as active or inactive ingredients) are present as separate components. The composition or dosage forms wherein the ingredients do not come as a mixture, but come as separate components, such composition/dosage forms may be administered in several ways. In one possible way, the ingredients may be mixed in the desired proportions and the mixture is reconstituted in suitable reconstitution diluent and then administered as required. Alternatively, the components or the ingredients (active or inert) may be separately administered (simultaneously or one after the other) in appropriate proportion so as to achieve the same or equivalent therapeutic level or effect as would have been achieved by administration of the equivalent mixture.

In some embodiments, pharmaceutical compositions according to the invention are formulated into a dosage form such that a compound of Formula (I) or a pharmaceutically acceptable salt thereof, and the beta-lactamase inhibitor or a pharmaceutically acceptable salt thereof, are present in the composition as admixture or as a separate components. In some other embodiments, pharmaceutical compositions according to the invention are formulated into a dosage form such that a compound of Formula (I) or a pharmaceutically acceptable salt thereof, and the beta-lactamase inhibitor or a pharmaceutically acceptable salt thereof, are present in the composition as separate components.

In another general aspect, pharmaceutical compositions according to the invention are used in treatment or prevention of a bacterial infection.

In yet another general aspect, there are provided methods for treating or preventing a bacterial infection in a subject, said method comprising administering to said subject effective amount of a pharmaceutical composition according to the invention.

In case of dosage forms wherein the compound of Formula (I) or a pharmaceutically acceptable salt thereof, and the beta-lactamase inhibitor or a pharmaceutically acceptable salt thereof, are present in the composition as separate components; the compound of Formula (I) or a pharmaceutically acceptable derivative thereof may be administered before, after or simultaneously with the administration of the beta-lactamase inhibitor or a pharmaceutically acceptable salt thereof.

In another general aspect, there are provided methods for treating or preventing bacterial infections in a subject, said methods comprising administering to said subject an effective amount of: (a) at least one beta-lactamase inhibitor or a pharmaceutically acceptable salt thereof, and (b) a compound of Formula (I):

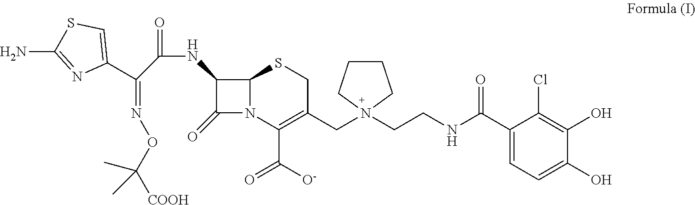

or a pharmaceutically acceptable salt thereof.

Individual amounts of the beta-lactamase inhibitor or a pharmaceutically acceptable salt thereof, and the compound of Formula (I) or a pharmaceutically acceptable salt thereof administered may vary depending on clinical requirements. In some embodiments, the beta-lactamase inhibitor or a pharmaceutically acceptable salt thereof is administered in an amount from about 0.01 gram to about 10 gram. In some other embodiments, the compound of Formula (I) or a pharmaceutically acceptable salt thereof is administered in an amount from about 0.01 gram to about 10 gram. In some other embodiments, the beta-lactamase inhibitor, or a pharmaceutically acceptable salt thereof is administered in an amount from about 0.125 gram to about 4 gram per gram of the compound of Formula (I) or a pharmaceutically acceptable salt thereof.

In some embodiments, in the methods according to the invention, the beta-lactamase inhibitor, or a pharmaceutically acceptable salt thereof, and the compound of Formula (I), or a pharmaceutically acceptable salt thereof, are administered in any of the following amounts:

(i) about 1 gram of the compound of Formula (I) or a pharmaceutically acceptable salt thereof, and about 0.25 gram of the beta-lactamase inhibitor, or a pharmaceutically acceptable salt thereof;

(ii) about 1 gram of the compound of Formula (I) or a pharmaceutically acceptable salt thereof, and about 0.50 gram of the beta-lactamase inhibitor, or a pharmaceutically acceptable salt thereof;

(iii) about 1 gram of the compound of Formula (I) or a pharmaceutically acceptable salt thereof, and about 1 gram of the beta-lactamase inhibitor, or a pharmaceutically acceptable salt thereof;

(iv) about 1 gram of the compound of Formula (I) or a pharmaceutically acceptable salt thereof, and about 2 gram of the beta-lactamase inhibitor, or a pharmaceutically acceptable salt thereof;

(v) about 2 gram of the compound of Formula (I) or a pharmaceutically acceptable salt thereof, and about 0.25 gram of the beta-lactamase inhibitor, or a pharmaceutically acceptable salt thereof;

(vi) about 2 gram of the compound of Formula (I) or a pharmaceutically acceptable salt thereof, and about 0.5 gram of the beta-lactamase inhibitor, or a pharmaceutically acceptable salt thereof;

(vii) about 2 gram of the compound of Formula (I) or a pharmaceutically acceptable salt thereof, and about 1 gram of the beta-lactamase inhibitor, or a pharmaceutically acceptable salt thereof;

(viii) about 2 gram of the compound of Formula (I) or a pharmaceutically acceptable salt thereof, and about 2 gram of the beta-lactamase inhibitor, or a pharmaceutically acceptable salt thereof;

(ix) about 0.5 gram of the compound of Formula (I) or a pharmaceutically acceptable salt thereof, and about 1 gram of the beta-lactamase inhibitor, or a pharmaceutically acceptable salt thereof; or

(x) about 0.5 gram of the compound of Formula (I) or a pharmaceutically acceptable salt thereof, and about 2 gram of the beta-lactamase inhibitor, or a pharmaceutically acceptable salt thereof.

In some embodiments, in the methods according to the invention, a compound of Formula (I) or a stereoisomer or a pharmaceutically acceptable salt thereof is administered before, after or simultaneously with the administration of the beta-lactamase inhibitor or a pharmaceutically acceptable salt thereof.

In the methods according to the invention, the pharmaceutical composition and/or other pharmaceutically active ingredients disclosed herein may be administered by any appropriate method, which serves to deliver the composition, or its constituents, or the active ingredients to the desired site. The method of administration can vary depending on various factors, such as for example, the components of the pharmaceutical composition and the nature of the active ingredients, the site of the potential or actual infection, the microorganism (e.g. bacteria) involved, severity of infection, age and physical condition of the subject. Some non-limiting examples of administering the composition to a subject according to this invention include oral, intravenous, topical, intrarespiratory, intraperitoneal, intramuscular, parenteral, sublingual, transdermal, intranasal, aerosol, intraocular, intratracheal, intrarectal, vaginal, gene gun, dermal patch, eye drop, ear drop or mouthwash. In some embodiments, the compositions or one or more active ingredients according to the invention are administered parenterally.

A wide variety of bacterial infections can be treated or prevented using compositions and methods according to the invention. Typical, non-limiting examples of bacterial infections that can be treated or prevented using methods and/or pharmaceutical compositions according to the invention include E. coli infections, Yersinia pestis (pneumonic plague), staphylococcal infection, mycobacteria infection, bacterial pneumonia, Shigella dysentery, Serratia infections, Candida infections, Cryptococcal infection, anthrax, tuberculosis or infections caused by Pseudomonas aeruginosa, Acinetobacter baumannii or methicillin resistant Staphylococcus aurues (MRSA) etc.

The pharmaceutical compositions and methods according to the invention are useful in treatment or prevention of several infections, including for example, skin and soft tissue infections, febrile neutropenia, urinary tract infection, intraabdominal infections, respiratory tract infections, pneumonia (nosocomial), bacteremia meningitis, surgical infections and the like.

In some embodiments, pharmaceutical compositions and methods according to the invention are used in treatment or prevention of infections caused by resistant bacteria. In some other embodiments, the compositions and methods according to the invention are used in treatment or prevention of infections caused by bacteria producing one or more beta-lactamase enzymes.

In general, the pharmaceutical compositions and methods disclosed herein are also effective in preventing or treating infections caused by bacteria that are considered to be less or not susceptible to one or more of known antibacterial agents or their known compositions. Some non-limiting examples of such bacteria known to have developed resistance to various antibacterial agents include Acinetobacter, Escherichia coli, Pseudomonas aeruginosa, Staphylococcus aureus, Enterobacter, Klebsiella, Citrobacter and a like.

The duration of treatment may depend on the nature of the active ingredients, the site of the potential or actual infection, the microorganism (e.g. bacteria) involved, severity of infection, age and physical condition of the subject. In some embodiments, the treatment may last between 1 to 14 days. In other embodiments, the treatment may last between 3 to 7 days. In some other embodiments, the treatment may last for 1 day, 3 days, 5 days, 7 days, 10 days or 14 days.

### EXAMPLES

The following examples illustrate embodiments of the invention that are presently best known. However, it is to be understood that the following are only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical embodiments of the invention.

In general, the antibacterial activity of compositions according to invention was studied by performing time kill studies. In a typical time kill study, the freshly grown cultures were diluted to the required cell density (initial starting inoculum) in cation adjusted Muller Hinton broth medium (BD, USA). The antibacterial agents (either alone or in combination) at the required concentrations were added into the culture-containing medium. The samples were incubated under shaking condition (120 rpm) at 37° C. Enumeration of viable bacterial count was done every 2 hour by diluting in normal saline and plating on to the Tryptic Soya Agar plates (BD, USA). The plates were incubated for 24 hours to arrive at a viable bacterial count. The results are expressed in terms of log₁₀ CFU/ml. In general, the decrease of 1 log₁₀ CFU/ml corresponds to 90% killing of bacteria. Similarly, 2 log₁₀ CFU/ml reductions indicates to 99% killing of bacteria and 3 log₁₀ CFU/ml reductions is equal to 99.9% killing of bacteria.

### Example 1

### Synthesis of Compound of Formula (I)

### Step-1: Preparation of Intermediate (1)

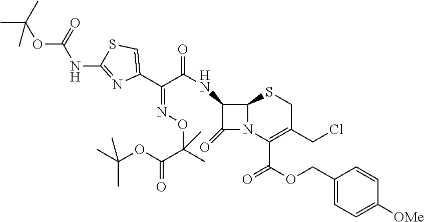 [SMILES: COc1ccc(COC(=O)C2=C(CCl)CS[C@@H]3[C@H](NC(=O)/C(=N\OC(C)(C)C(=O)OC(C)(C)C)c4csc(NC(=O)OC(C)(C)C)n4)C(=O)N23)cc1]

To the clear solution of (Z)-2[(2-tert-butoxycarbonyl amino-thiazol-4-yl)-carboxy-methyleneaminooxy]2-methyl-propionic acid tert-butyl ester (30 gm, 69.93 mmol) in N,N-dimethyl acetamide (300 ml) was charged triethylamine (17.68 ml, 125.87 mmol) under stirring. The reaction mixture was cooled to −15° C. Methane sulfonyl chloride (12.01 gm, 104. 89 mmol) was charged to this cooled reaction mixture via addition funnel while maintaining temperature at about −15° C. The reaction mixture was stirred for 30 minutes at −15° C. after the addition. To the reaction mixture was charged (6R,7S)-4-methoxybenzyl-7-amino-3-chloromethyl-8-oxo-5-thia-1-azabicyclo[4.2.0]oct-2-ene-2-carboxylate hydrochloride salt (28.25 gm, 69.93 mmol) along with N-methyl morpholine (15.5 ml, 139.86 mmol). The reaction mixture was stirred further for 1 hour at −15° C. and the reaction progress was monitored using TLC. After completion of reaction, ethyl acetate (1.2 L) was charged followed by 1N aqueous hydrochloric acid (1.2 L) under stirring and cooling was removed to warm up reaction mixture to room temperature. Layers were separated and organic layer was washed with saturated aqueous sodium bicarbonate solution (500 ml) followed by brine (500 ml). Organic layer was dried over sodium sulphate and was evaporated under vacuum to provide a crude mass. It was purified using silica gel column chromatography (60-120 mesh, 30% ethyl acetate in hexane) to provide 38 gm of intermediate (1).

Analysis:

¹H NMR (CDCl₃) δ ppm: 8.29 (br s, 1H), 8.17 (d, 1H), 7.35 (d, 2H), 7.31 (s, 1H), 6.91 (d, 2H), 6.21 (dd, 1H), 5.23 (dd, 2H), 5.05 (d, 1H), 4.55 (d, 1H), 4.46 (d, 1H), 3.82 (s, 3H), 3.65 (d, 1H), 3.48 (d, 1H), 1.62 (s, 3H), 1.59 (s, 3H), 1.53 (s, 9H), 1.45 (s, 9H).

### Step-2: Preparation of Intermediate (2)

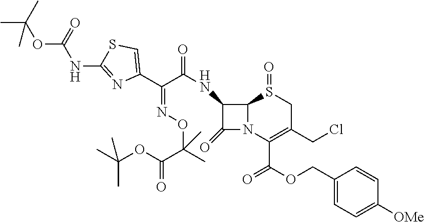

The solution of intermediate 1 (45 gm, 57.76 mmol) in dichloromethane (450 ml) was cooled to about −40° C. and m-chloroperbenzoic acid (18 gm, 57.76 mmol) was added in three lots at −40° C. under stirring. The mixture was stirred for 30 minutes and allowed to warm at −20° C. As TLC showed complete conversion, 5% aqueous sodium thiosulfate solution (1.2 L) was added at −15° C. under stirring. The mixture was allowed to warm at room temperature and was charged with ethyl acetate (1.5 L) and stirred for 30 minutes and layers were separated. Organic layer was washed with saturated aqueous sodium bicarbonate solution (1 L) followed by brine (500 ml). Organic layer was dried over sodium sulphate and evaporated under vacuum to provide 46 gm of intermediate (2).

Analysis:

¹H NMR (CDCl₃) δ ppm: 8.48 (br s, 1H), 7.89 (d, 1H), 7.34 (d, 2H), 7.29 (s, 1H), 6.92 (d, 2H), 6.21 (dd, 1H), 5.27 (dd, 2H), 5.04 (br d, 1H), 4.58 (d, 1H), 4.23 (d, 1H), 3.83 (s, 3H), 3.82 (d, 1H), 3.43 (d, 1H), 1.60 (s, 3H), 1.58 (s, 3H), 1.53 (9H) 1.42 (s, 9H).

### Step-3: Preparation of Intermediate (3)

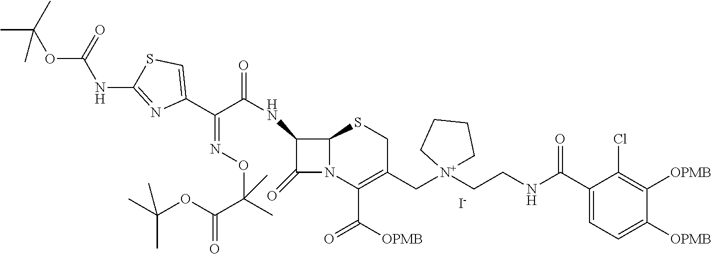

Part-1: To the clear solution of intermediate 2 (35 gm, 44.02 mmol) in tetrahydrofuran (350 ml) was charged potassium iodide (14.61 gm, 88.05 mmol) under stirring at 25° C. The suspension was stirred for 5 hours at the same temperature and the reaction was monitored using mass spectroscopy. After completion of the reaction ethyl acetate (600 ml) was added to the reaction mixture followed by 5% aqueous sodium thiosulphate (600 ml). The reaction mixture was stirred for 15 minutes and layers were separated. Organic layer was washed with demineralised water (500 ml) followed by brine (500 ml). Organic layer was dried over sodium sulphate and evaporated to dryness under vacuum to provide 38 gm of corresponding iodo-methyl intermediate.

Part-2: To the iodo-methyl intermediate obtained (37.24 gm, 41.98 mmol) in N,N-dimethylformamide (35 ml) was added 2-chloro-3,4-di-(4-methoxybenzyloxy)-N-(pyrrolidin-1-ylethyl)-benzamide (22 gm, 42.98 mmol). The thick mass was stirred at 25° C. for 15 hours and the reaction was monitored using mass spectroscopy. Potassium iodide (48.78 gm, 293.8 mmol) was charged to the reaction mass under stirring at 25° C. The reaction mixture was cooled to −40° C. and acetyl chloride (12 ml, 167.9 mmol) was added. After completion of the reaction ethyl acetate (1.2 L) followed by demineralised water (1.2 L) was added to the reaction mass at 0° C. Layers were separated and organic layer was washed with demineralised water (500 ml) followed by brine (500 ml). Organic layer was dried over sodium sulphate and was evaporated to dryness under vacuum to obtain quaternary intermediate (3) as iodide salt.

### Step-4: Preparation Compound of Formula (I)

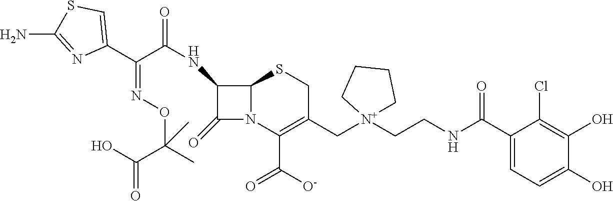 [SMILES: CC(C)(O/N=C(\C(=O)N[C@@H]1C(=O)N2C(C(=O)[O-])=C(C[N+]3(CCNC(=O)c4ccc(O)c(O)c4Cl)CCCC3)CS[C@H]12)c1csc(N)n1)C(=O)O]

Compound (3) (30 gm, 21.5 mmol) was dissolved in dichloromethane (300 ml) and anisole (30 gm, mmol) was added under stirring at 25° C. The mixture was cooled to −40° C. and 2M aluminium chloride solution in nitromethane (150 ml) was added over 45 minutes at −40° C. As addition was completed reaction mixture was stirred for 1 hour at 0° C. To the reaction mixture 2M aqueous hydrochloric acid (750 ml) and acetonitrile (750 ml) were added and the stirring was continued for 15 minutes. Di-isopropyl ether (1.5 L) was charged to the reaction mixture and the reaction mass was stirred for 15 minutes at 25° C., and the layers were separated. Aqueous layer was washed with additional di-isopropyl ether (500 ml). HP-21 resin (150 gm) was charged to the aqueous layer. The aqueous layer along with resin was loaded on a resin HP-21 column. The column was eluted with demineralised water till pH of eluent became neutral. Then the column was eluted with 10% acetonitrile in water mixture. Finally the column was eluted with 20% acetonitrile in water mixture. Evaporation of required fractions below 40° C. under vacuum provided 5.5 gm of crude compound (I). The crude compound (I) was purified by dissolving in acetonitrile (200 ml) and demineralised water (200 ml) mixture followed by addition of HP-21 resin (200 gm). The slurry thus obtained was loaded on HP-21 resin column. The column was eluted first with demineralised water (3 L) followed by 10% acetonitrile in water mixture (2 L) then followed by 20% acetonitrile in water mixture till complete pure compound from the column is eluted. Pure fractions were collected and lyophilized under vacuum to provide titled compound (I) in pure form.

Analysis:

¹H NMR (DMSO d₆) 6 ppm: 12.5 (br s, 2H), 9.42 (br s, 1H), 8.41 (br t, 1H), 7.28 (br s, 3H), 6.78 (s, 2H), 6.73 (s, 1H), 5.73 (dd, 1H), 5.15 (d, 1H), 5.08 (br d, 1H), 3.71-3.91 (m, 4H), 3.21-3.60 (m, 7H), 1.95-2.19 (m, 4H) 1.76 (s, 3H), 1.44 (s, 3H).

HPLC purity: 90.80%

### Example 2

The results on the antibacterial activity of the compound of Formula (I) alone and in combination with various beta-lactamase inhibitors against K. pneumoniae B-88 are given in Table 1. K. pneumoniae B-88 produces resistant metallo beta-lactamase enzymes. As can be seen from the data in Table 1, Compound (I), (B), (C), (D) and (E) alone could not reduce the bacterial count. Surprisingly, a combination of Compound (I) with Compounds (B), (C), (D) and (E) significantly reduced the bacterial counts. For example, a combination of a compound (I) (0.25 mcg/ml or 0.5 mcg/ml) and Compound (C) (2 mcg/ml) exhibited potent antibacterial activity with 5.28 log₁₀ reduction in bacterial count after 24 hours.

TABLE 1
Antibacterial activity of compound of Formula (I) alone or in combination with various beta-
lactamase inhibitors against K. pneumoniae B-88 producing metallo beta-lactamase enzymes.

| | | | | | | | Change in Log₁₀ |
|---|---|---|---|---|---|---|---|
| | | Bacterial count (Log₁₀ CFU/ml) | | | | | CFU/ml over 0 |
| | | 0 | 2 | 4 | 6 | 24 | hour count after |
| Sr. | Combination | hours | hours | hours | hours | hours | 24 hours* |
| 1. | Control (No active ingredient) | 7.28 | 8.27 | 9.04 | 9.18 | 9.40 | 2.12 |
| 2. | Compound of Formula (I) (0.25 mcg/ml) | 7.28 | 6.74 | 6.60 | 6.70 | 6.81 | −0.47 |
| 3. | Compound of Formula (I) (0.5 mcg/ml) | 7.28 | 6.18 | 5.85 | 6.30 | 6.30 | −0.98 |
| 4. | Compound of Formula (I) (0.5 mcg/ml) + | 7.28 | 6.18 | 5.85 | 6.30 | 6.30 | −0.98 |
| | Compound (B) (2 mcg/ml) | | | | | | |
| 5. | Compound of Formula (I) (0.25 mcg/ml) + | 7.28 | 6.30 | 4.60 | 4.26 | 2.00 | −5.28 |
| | Compound (C) (2 mcg/ml) | | | | | | |
| 6. | Compound of Formula (I) (0.5 mcg/ml) + | 7.28 | 6.15 | 4.65 | 4.08 | 2.00 | −5.28 |
| | Compound (C) (2 mcg/ml) | | | | | | |
| 7. | Compound of Formula (I) (0.25 mcg/ml) + | 7.28 | 6.15 | 4.18 | 4.20 | 3.30 | −3.98 |
| | Compound (D) (2 mcg/ml) | | | | | | |
| 8. | Compound of Formula (I) (0.5 mcg/ml) + | 7.28 | 5.81 | 4.40 | 3.90 | 2.00 | −5.28 |
| | Compound (D) (2 mcg/ml) | | | | | | |
| 9. | Compound of Formula (I) (0.25 mcg/ml) + | 7.28 | 6.20 | 4.70 | 4.08 | 3.40 | −3.88 |
| | Compound (E) (2 mcg/ml) | | | | | | |
| 10. | Compound of Formula (I) (0.5 mcg/ml) + | 7.28 | 5.65 | 4.26 | 3.78 | 2.00 | −5.28 |
| | Compound (E) (2 mcg/ml) | | | | | | |
| 11. | Imipenem (8 mcg/ml) | 7.28 | 8.04 | 8.65 | 9.20 | 9.30 | 2.02 |
| 12. | Imipenem (1 mcg/ml) + | 7.28 | 6.15 | 5.08 | 5.34 | 5.60 | −1.68 |
| | Ethylenediaminetetraacetate (200 mcg/ml) | | | | | | |
| 13. | Compound of Formula (I) (0.5 mcg/ml) + | 7.28 | 6.78 | 5.26 | 5.08 | 6.04 | −1.28 |
| | Ethylenediaminetetraacetate (200 mcg/ml) | | | | | | |
| 14. | Compound (B) (2 mcg/ml) | 7.28 | 8.27 | 9.04 | 9.18 | 9.40 | 2.12 |
| 15. | Compound (C) (2 mcg/ml) | 7.28 | 7.60 | 8.26 | 8.18 | 8.54 | 1.26 |
| 16. | Compound (D) (2 mcg/ml) | 7.28 | 7.60 | 8.26 | 8.18 | 8.54 | 1.26 |
| 17. | Compound (E) (2 mcg/ml) | 7.28 | 7.60 | 8.26 | 8.18 | 8.54 | 1.26 |

*Note:
Positive value indicates increase in bacterial count over 0 hour count; and negative value indicates reduction in bacterial count over 0 hour count.

### Example 3

The results on the antibacterial activity of the compound of Formula (I) alone and in combination with various beta-lactamase inhibitors against E. coli 7MP are given in Table 2. E. coli 7MP produces Class A and Class C beta-lactamase enzymes. As can be seen from the data in Table 2, Compound (I), (B), (C), (D) and (E) alone could not reduce the bacterial count. However, it was observed that combination of Compound (I), with Compounds (B), (C), (D) and (E) significantly reduced the bacterial counts. For example, a combination of a compound of Formula (I) (1 mcg/ml) and Compound (B) (4 mcg/ml) exhibited potent antibacterial activity with 6.08 log₁₀ reduction in bacterial count after 24 hours.

TABLE 2
Antibacterial activity of Compound (I) alone or in combination against
E. coli 7MP producing Class A and Class C beta-lactamase enzymes.

| | | | | | | | Change in Log₁₀ |
|---|---|---|---|---|---|---|---|
| | | Bacterial count (Log₁₀ CFU/ml) | | | | | CFU/ml over 0 |
| | | 0 | 2 | 4 | 6 | 24 | hour count after |
| Sr. | Combination | hours | hours | hours | hours | hours | 24 hours* |
| 1. | Control (No active ingredient) | 7.08 | 7.90 | 9.23 | 9.30 | 9.36 | 2.28 |
| 2. | Compound of Formula (I) (0.25 mcg/ml) | 7.08 | 7.60 | 9.11 | 9.20 | 9.20 | 2.12 |
| 3. | Compound of Formula (I) (0.5 mcg/ml) | 7.08 | 7.18 | 7.74 | 9.18 | 9.20 | 2.12 |
| 4. | Compound of Formula (I) (1 mcg/ml) + | 7.08 | 5.15 | 4.08 | 3.40 | 1.00 | −6.08 |
| | Compound (B) (4 mcg/ml) | | | | | | |
| 5. | Compound of Formula (I) (0.25 mcg/ml) + | 7.08 | 4.98 | 3.74 | 3.27 | 2.00 | −5.08 |
| | Compound (C) (2 mcg/ml) | | | | | | |
| 6. | Compound of Formula (I) (0.5 mcg/ml) + | 7.08 | 4.93 | 3.81 | 3.00 | 2.00 | −5.08 |
| | Compound (C) (2 mcg/ml) | | | | | | |
| 7. | Compound of Formula (I) (0.25 mcg/ml) + | 7.08 | 5.32 | 4.26 | 3.54 | 2.00 | −5.08 |
| | Compound (D) (2 mcg/ml) | | | | | | |
| 8. | Compound of Formula (I) (0.5 mcg/ml) + | 7.08 | 5.30 | 4.65 | 3.40 | 2.00 | −5.08 |
| | Compound (D) (2 mcg/ml) | | | | | | |
| 9. | Compound of Formula (I) (0.25 mcg/ml) + | 7.08 | 6.65 | 5.11 | 4.26 | 2.00 | −5.08 |
| | Compound (E) (2 mcg/ml) | | | | | | |
| 10. | Compound of Formula (I) (0.5 mcg/ml) + | 7.08 | 5.48 | 4.04 | 3.95 | 2.00 | −5.08 |
| | Compound (E) (2 mcg/ml) | | | | | | |
| 11. | Compound (B) (4 mcg/ml) | 7.08 | 7.90 | 9.23 | 9.30 | 9.36 | 2.28 |
| 12. | Compound (C) (2 mcg/ml) | 7.08 | 6.30 | 4.93 | 4.23 | 3.54 | −3.54 |
| 13. | Compound (C) (2 mcg/ml) + | 7.08 | 6.85 | 4.93 | 5.08 | 6.78 | −0.3 |
| | Apoterin (20 mM) | | | | | | |
| 14. | Compound (D) (2 mcg/ml) | 7.08 | 7.65 | 7.30 | 7.78 | 7.30 | 0.22 |
| 15. | Compound (E) (2 mcg/ml) | 7.08 | 8.02 | 9.15 | 9.11 | 9.30 | 2.22 |

*Note:
Positive value indicates increase in bacterial count over 0 hour count; and negative value indicates reduction in bacterial count over 0 hour count.

### Example 4

The results on the antibacterial activity of the compound of Formula (I) alone and in combination with various beta-lactamase inhibitors against A. baumanni 13301 are given in Table 3. A. baumanni 13301 produces carbapenem hydrolysing beta-lactamase enzymes. As can be seen from the data in Table 3, Compound of Formula (I), (B), (C), (D) or (E) alone could not reduce the bacterial count. However, a combination of Compound of Formula (I), with Compound (B), (C), (D) or (E) significantly reduced the bacterial counts. For example, a combination of compound of Formula (I) (4.0 mcg/ml) with Compound (B) (4 mcg/ml) exhibited potent antibacterial activity with 3.88 log₁₀ reduction in bacterial count after 24 hours.

The results given in the Tables 1, 2 and 3, clearly demonstrate the surprisingly potent synergistic antibacterial activity of the combination comprising compound of Formula (I) and a beta-lactamase inhibitor, even against highly resistant bacterial strains producing extended spectrum beta-lactamase enzymes including metallo beta-lactamase enzymes, Class C beta-lactamase enzymes and carbapenem hydrolyzing beta-lactamase enzymes. Thus, combination of a compound of Formula (I) and a beta-lactamase inhibitor has beneficial effect in inhibiting highly resistant bacterial strains demonstrating the noteworthy therapeutic advance in the treatment of infections caused by resistant bacteria.

TABLE 3
Antibacterial activity of compound of Formula (I) alone or in combination against
A. baumanni 13301 producing Carbapenem hydrolysing beta-lactamase enzymes.

| | | | | | | | | Change in Log₁₀ |
|---|---|---|---|---|---|---|---|---|
| | | Bacterial count (Log₁₀ CFU/ml) | | | | | | CFU/ml over 0 |
| | | 0 | 2 | 4 | 6 | 8 | 24 | hour count after |
| Sr. | Combination | hours | hours | hours | hours | hours | hours | 24 hours* |
| 1. | Control (No active ingredient) | 6.88 | 8.04 | 8.74 | 8.90 | 9.36 | 9.04 | 2.16 |
| 2. | Compound of Formula (I) (4 mcg/ml) | 6.88 | 4.85 | 4.02 | 5.18 | 7.5 | 9.10 | 2.22 |
| 3. | Compound of Formula (I) (4 mcg/ml) + | 6.88 | 6.81 | 5.74 | 4.40 | 3.13 | 3.00 | −3.88 |
| | Compound (B) (4 mcg/ml) | | | | | | | |
| 4. | Compound of Formula (I) (4 mcg/ml) + | 6.88 | 6.20 | 4.65 | 3.30 | 2.95 | 6.30 | −0.58 |
| | Compound (C) (4 mcg/ml) | | | | | | | |
| 5. | Compound of Formula (I) (4 mcg/ml) + | 6.88 | 6.90 | 6.02 | 4.78 | 3.68 | 7.70 | 0.82 |
| | Compound (D) (4 mcg/ml) | | | | | | | |
| 6. | Compound of Formula (I) (4 mcg/ml) + | 6.88 | 6.93 | 5.85 | 4.65 | 3.35 | 8.93 | 2.05 |
| | Compound (E) (4 mcg/ml) | | | | | | | |
| 7. | Compound (B) (4 mcg/ml) | 6.88 | 8.04 | 8.74 | 8.90 | 9.20 | 9.04 | 2.16 |
| 8. | Compound (C) (4 mcg/ml) | 6.88 | 8.13 | 8.85 | 9.19 | 9.02 | 9.23 | 2.35 |
| 9. | Compound (C) (4 mcg/ml) + | 6.88 | 8.06 | 8.98 | 9.60 | 9.60 | 9.08 | 2.17 |
| | Apoterin (20 mM) | | | | | | | |
| 10. | Compound (D) (4 mcg/ml) | 6.88 | 7.98 | 8.60 | 9.00 | 9.04 | 8.95 | 2.07 |
| 11. | Compound (E) (4 mcg/ml) | 6.88 | 8.04 | 8.74 | 8.90 | 9.20 | 9.04 | 2.16 |

*Note:
Positive value indicates increase in bacterial count over 0 hour count; and negative value indicates reduction in bacterial count over 0 hour count.

### Example 5

Manufacturing Procedure:

The compound of Formula (I), Compound (C), microcrystalline cellulose, croscarmellose sodium were weighed, sifted, and mixed in a Rapid Mixer Granulator. The above mass was granulated by spraying aqueous solution of povidone. The granules were dried in a fluidized bed drier, sifted and milled. The resulting granules were blended with sifted microcrystalline cellulose, croscarmellose sodium, talc and magnesium stearate. The lubricated granules were compressed into tablets using suitable tooling. The tablets were coated with aqueous dispersion of Opadry®. The composition is shown in Table 4.

TABLE 4
Pharmaceutical compositions according to the invention

| | | mg/Tablet | |
|---|---|---|---|
| | | Formu- | Formu- |
| Sr. | Ingredients | lation 1 | lation 2 |

INTRAGRANULAR

| | | | |
|---|---|---|---|
| 1 | Compound of Formula (I) | 1000.0 | 1000.0 |
| 2 | Compound (C) | 250.0 | 500.0 |
| 3 | Microcrystalline Cellulose (Avicel PH | 80.0 | 160.0 |
| | 101) | | |
| 4 | Croscarmesllose Sodium (Ac-Di-Sol) | 7.0 | 14.0 |
| 5 | Povidone K30 (Kollidone K30) | 8.75 | 17.50 |
| 6 | Purified water USP | q.s. | q.s. |

EXTRAGRANULAR

| | | | |
|---|---|---|---|
| 7 | Microcrystalline cellulose (Avicel PH | 31.25 | 62.50 |
| | 102) | | |
| 8 | Croscarmesllose Sodium (Ac-Di-Sol) | 13.0 | 26.0 |
| 9 | Talc | 3.50 | 7.00 |
| 10 | Magnesium stearate | 3.0 | 6.0 |

| | | |
|---|---|---|
| CORE TABLET (mg) | 1300.0 | 1800.0 |

FILM COATING

| | | | |
|---|---|---|---|
| 11 | Opadry ®. Yellow (03B28796) | 10.5 | 21.0 |
| 12 | Purified Water USP | q.s. | q.s. |

| | | |
|---|---|---|
| Total (Coated Tablet Weight) mg | 1310.50 | 1821.0 |

### Example 6

The results on the antibacterial activity of the compound of Formula (I) alone and in combination with beta-lactamase inhibitors, against E. coli 7MP are given in Table 5. E. coli 7MP produces Class A and C beta-lactamase enzymes. As can be seen from the data in Table 5, Compound of Formula (I), and the beta-lactamase inhibitor selected from tazobactam, clavulanic acid or avibactam when used alone, did not reduce the bacterial count throughout the duration of the study. However, surprisingly, it was observed that combination of Compound of Formula (I), and the beta-lactamase inhibitor selected from tazobactam, clavulanic acid or avibactam significantly reduced the bacterial counts throughout the duration of the study. For example, a combination of a compound of Formula (I) (1 mcg/ml) and Avibactam (4 mcg/ml) exhibited potent antibacterial activity with 3.78 log₁₀ reduction in bacterial count after 8 hours.

TABLE 5
Antibacterial activity of compound of Formula (I) alone or in combination with beta-lactamase
inhibitor against E. coli 7 MP producing Class A & C beta-lactamase enzymes.

| | | | | | | | Change in Log₁₀ |
|---|---|---|---|---|---|---|---|
| | | Bacterial count (Log₁₀ CFU/ml) | | | | | CFU/ml over 0 |
| | | 0 | 2 | 4 | 6 | 8 | hour count after |
| Sr. | Combination | hours | hours | hours | hours | hours | 8 hours* |
| 1. | Control (No active ingredient) | 7.08 | 7.90 | 9.23 | 9.30 | 9.36 | 2.28 |
| 2. | Compound of Formula (I) (1 mcg/ml) | 7.08 | 7.15 | 6.60 | 7.90 | 8.65 | 1.57 |
| 3. | Compound of Formula (I) (1 mcg/ml) + | 7.08 | 5.88 | 4.85 | 3.85 | 3.60 | −3.48 |
| | Tazobactam (4 mcg/ml) | | | | | | |
| 4. | Compound of Formula (I) (1 mcg/ml) + | 7.08 | 6.95 | 5.54 | 5.40 | 6.65 | −0.43 |
| | Clavulanic acid (4 mcg/ml) | | | | | | |
| 5. | Compound of Formula (I) (1 mcg/ml) + | 7.08 | 5.10 | 3.95 | 3.40 | 3.30 | −3.78 |
| | Avibactam (4 mcg/ml) | | | | | | |
| 6. | Tazobactam (4 mcg/ml) | 7.08 | 7.90 | 9.23 | 9.30 | 9.36 | 2.28 |
| 7. | Clavulanic acid (4 mcg/ml) | 7.08 | 7.90 | 9.23 | 9.30 | 9.36 | 2.28 |
| 8. | Avibactam (4 mcg/ml) | 7.08 | 7.90 | 9.23 | 9.30 | 9.36 | 2.28 |

*Note:
Positive value indicates increase in bacterial count over 0 hour count; and negative value indicates reduction in bacterial count over 0 hour count.

The results given in the Tables 5, clearly demonstrate the surprisingly potent synergistic antibacterial activity of the combination comprising compound of Formula (I) and at least one beta-lactamase inhibitor selected from tazobactam, clavulanic acid or avibactam, even against highly resistant bacterial strains producing extended spectrum beta-lactamase enzymes including Class A and Class C beta-lactamase enzymes. Thus, combination of a compound of Formula (I) or a pharmaceutically acceptable derivative thereof and at least one beta-lactamase inhibitor or a pharmaceutically acceptable derivative thereof has tremendous beneficial effect in inhibiting highly resistant bacterial strains demonstrating the noteworthy therapeutic advance in the treatment of infections caused by resistant bacteria.

### Example 7

Manufacturing Procedure:

The compound of Formula (I), potassium clavulanate, microcrystalline cellulose, croscarmellose sodium were weighed, sifted, and mixed in a Rapid Mixer Granulator. The above mass was granulated by spraying aqueous solution of povidone. The granules were dried in a fluidized bed drier, sifted and milled. The resulting granules were blended with sifted microcrystalline cellulose, croscarmellose sodium, talc and magnesium stearate. The lubricated granules were compressed into tablets using suitable tooling. The tablets were coated with aqueous dispersion of Opadry®. The composition is shown in Table 6.

TABLE 6
Pharmaceutical compositions according to the invention

| | | mg/Tablet | |
|---|---|---|---|
| | | Formu- | Formu- |
| Sr. | Ingredients | lation 1 | lation 2 |

INTRAGRANULAR

| | | | |
|---|---|---|---|
| 1 | Compound of Formula (I) | 1000.0 | 1000.0 |
| 2 | Potassium clavulanate | 250.0 | 500.0 |
| 3 | Microcrystalline Cellulose (Avicel PH | 80.0 | 160.0 |
| | 101) | | |
| 4 | Croscarmesllose Sodium (Ac-Di-Sol) | 7.0 | 14.0 |
| 5 | Povidone K30 (Kollidone K30) | 8.75 | 17.50 |
| 6 | Purified water USP | q.s. | q.s. |

EXTRAGRANULAR

| | | | |
|---|---|---|---|
| 7 | Microcrystalline cellulose (Avicel PH | 31.25 | 62.50 |
| | 102) | | |
| 8 | Croscarmesllose Sodium (Ac-Di-Sol) | 13.0 | 26.0 |
| 9 | Talc | 3.50 | 7.00 |
| 10 | Magnesium stearate | 3.0 | 6.0 |

| | | |
|---|---|---|
| CORE TABLET (mg) | 1300.0 | 1800.0 |

FILM COATING

| | | | |
|---|---|---|---|
| 11 | Opadry ®. Yellow (03B28796) | 10.5 | 21.0 |
| 12 | Purified Water USP | q.s. | q.s. |

| | | |
|---|---|---|
| Total (Coated Tablet Weight) mg | 1310.50 | 1821.0 |

1. A pharmaceutical composition comprising: (a) at least one beta-lactamase inhibitor, or a pharmaceutically acceptable salt thereof; and (b) a compound of Formula (I): 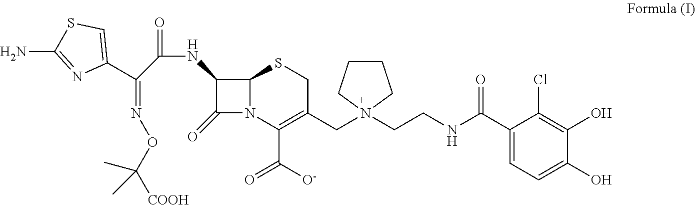
or a pharmaceutically acceptable salt thereof.

2. The pharmaceutical composition according to claim 1, wherein the beta-lactamase inhibitor is at least one selected from the group consisting of sulbactam, tazobactam, clavulanic acid, or avibactam.

3. The pharmaceutical composition according to claim 1, wherein the beta-lactamase inhibitor is a compound of Formula (II): 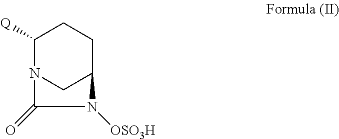
or a stereoisomer thereof,
wherein Q is:
(a) cyano;
(b) five to fourteen membered heteroaryl, optionally substituted with one or more of the following:
(i) —CO—NH₂,
(ii) five to fourteen membered heteroaryl,
(iii) three to seven membered heterocycloalkyl,
(iv) three to seven membered cycloalkyl,
(v) five to fourteen membered aryl, or
(vi) C₁-C₆ alkyl, optionally substituted with —NH₂ or three to seven membered heterocycloalkyl;
(c) —CO—NH—NH—CO—R₁;
(d) —CO—NH—O—R₁; or
(e) —CO—NH—R₁;
R₁ is
(a) hydrogen;
(b) three to seven membered heterocycloalkyl, optionally substituted with hydroxy group;
(c) C₁-C₆ alkyl, optionally substituted with: (i) three to seven membered heterocycloalkyl; (ii) one or more hydroxy groups; or (ii) one or more amino groups.

4. The pharmaceutical composition according to claim 1, wherein the beta-lactamase inhibitor is selected from a group consisting of: 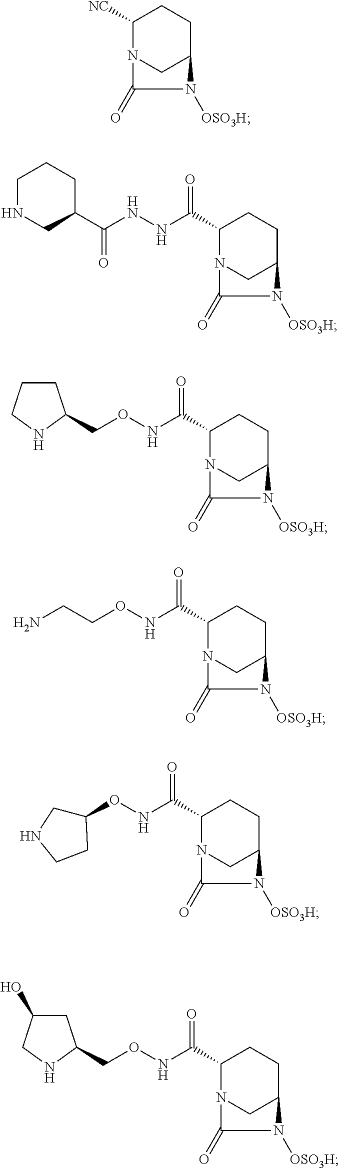 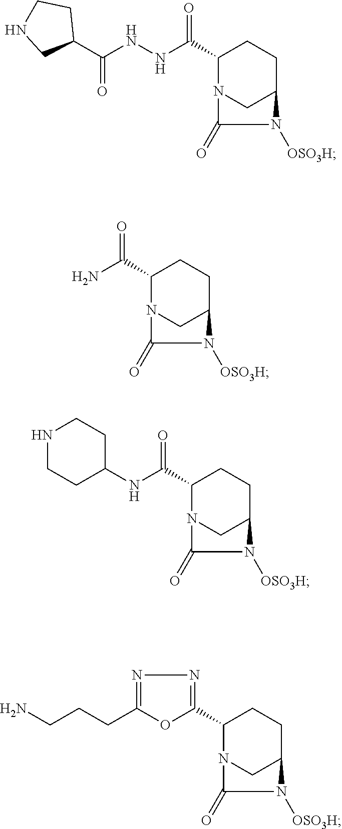
or a stereoisomer or a pharmaceutically acceptable salt thereof.

5. The pharmaceutical composition according to any one of the claims 1 to 4, wherein the beta-lactamase inhibitor or a pharmaceutically acceptable salt thereof is present in the composition in an amount from about 0.01 gram to about 10 gram.

6. The pharmaceutical composition according to any one of the claims 1 to 4, wherein the compound of Formula (I) or a pharmaceutically acceptable derivative thereof is present in the composition in an amount from about 0.01 gram to about 10 gram.

7. The pharmaceutical composition according to any one of the claims 1 to 4, wherein the beta-lactamase inhibitor, or a pharmaceutically acceptable salt thereof is present in the composition in an amount from about 0.125 gram to about 4 gram per gram of the compound of Formula (I) or a pharmaceutically acceptable salt thereof.

8. The pharmaceutical composition according to any one of the claims 1 to 4, comprising: (a) at least one beta-lactamase inhibitor, or a pharmaceutically acceptable salt thereof, and (b) a compound of Formula (I), or a pharmaceutically acceptable salt thereof, in any of the following amounts:
(i) about 1 gram of the compound of Formula (I) or a pharmaceutically acceptable salt thereof, and about 0.25 gram of the beta-lactamase inhibitor, or a pharmaceutically acceptable salt thereof;
(ii) about 1 gram of the compound of Formula (I) or a pharmaceutically acceptable salt thereof, and about 0.50 gram of the beta-lactamase inhibitor, or a pharmaceutically acceptable salt thereof;
(iii) about 1 gram of the compound of Formula (I) or a pharmaceutically acceptable salt thereof, and about 1 gram of the beta-lactamase inhibitor, or a pharmaceutically acceptable salt thereof;
(iv) about 1 gram of the compound of Formula (I) or a pharmaceutically acceptable salt thereof, and about 2 gram of the beta-lactamase inhibitor, or a pharmaceutically acceptable salt thereof;
(v) about 2 gram of the compound of Formula (I) or a pharmaceutically acceptable salt thereof, and about 0.25 gram of the beta-lactamase inhibitor, or a pharmaceutically acceptable salt thereof;
(vi) about 2 gram of the compound of Formula (I) or a pharmaceutically acceptable salt thereof, and about 0.5 gram of the beta-lactamase inhibitor, or a pharmaceutically acceptable salt thereof;
(vii) about 2 gram of the compound of Formula (I) or a pharmaceutically acceptable salt thereof, and about 1 gram of the beta-lactamase inhibitor, or a pharmaceutically acceptable salt thereof;
(viii) about 2 gram of the compound of Formula (I) or a pharmaceutically acceptable salt thereof, and about 2 gram of the beta-lactamase inhibitor, or a pharmaceutically acceptable salt thereof;
(ix) about 0.5 gram of the compound of Formula (I) or a pharmaceutically acceptable salt thereof, and about 1 gram of the beta-lactamase inhibitor, or a pharmaceutically acceptable salt thereof; or
(x) about 0.5 gram of the compound of Formula (I) or a pharmaceutically acceptable salt thereof, and about 2 gram of the beta-lactamase inhibitor, or a pharmaceutically acceptable salt thereof.

9. The pharmaceutical composition according to any of the claims 1 to 8, wherein the composition is formulated into a dosage form such that the compound of Formula (I) or a stereoisomer or a pharmaceutically acceptable derivative thereof, and the beta-lactamase inhibitor or a pharmaceutically acceptable salt, are present in the composition as admixture or as separate components.

10. The pharmaceutical composition according to any of the claims 1 to 7, wherein the composition is in the form of a powder or a solution.

11. The pharmaceutical composition according to claim 9, wherein the composition is in the form of a powder or a solution that can be reconstituted by addition of a compatible reconstitution diluent.

12. The pharmaceutical composition according to any one of the claims 1 to 11, for use in treatment or prevention of a bacterial infection.

13. A method for preventing or treating a bacterial infection in a subject, said method comprising administering to said subject an effective amount of a pharmaceutical composition according to any one of the claims 1 to 12.

14. A method for preventing or treating a bacterial infection in a subject, said method comprising administering to said subject an effective amount of: (a) at least one beta-lactamase inhibitor, or a pharmaceutically acceptable salt thereof; and (b) a compound of Formula (I): 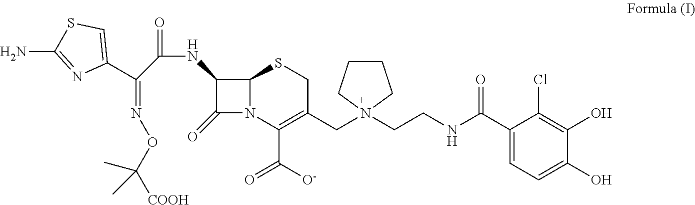
or a pharmaceutically acceptable salt thereof.

15. The method according to claim 14, wherein the beta-lactamase inhibitor is at least one selected from the group consisting of sulbactam, tazobactam, clavulanic acid, or avibactam.

16. The method according to claim 14, wherein the beta-lactamase inhibitor is a compound of Formula (II): 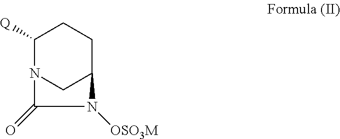
or a stereoisomer thereof,
wherein Q is:
(a) cyano;
(b) five to fourteen membered heteroaryl, optionally substituted with one or more of the following:
(i) —CO—NH₂,
(ii) five to fourteen membered heteroaryl,
(iii) three to seven membered heterocycloalkyl,
(iv) three to seven membered cycloalkyl,
(v) five to fourteen membered aryl, or
(vi) C₁-C₆ alkyl, optionally substituted with —NH₂ or three to seven membered heterocycloalkyl;
(c) —CO—NH—NH—CO—R₁;
(d) —CO—NH—O—R₁; or
(e) —CO—NH—R₁;
R1 is
(a) hydrogen;
(b) three to seven membered heterocycloalkyl, optionally substituted with hydroxy group;
(c) C₁-C₆ alkyl, optionally substituted with: (i) three to seven membered heterocycloalkyl; (ii) one or more hydroxy groups; or (ii) one or more amino groups.

17. The method according to claim 14, wherein the beta-lactamase inhibitor is selected from a group consisting of: 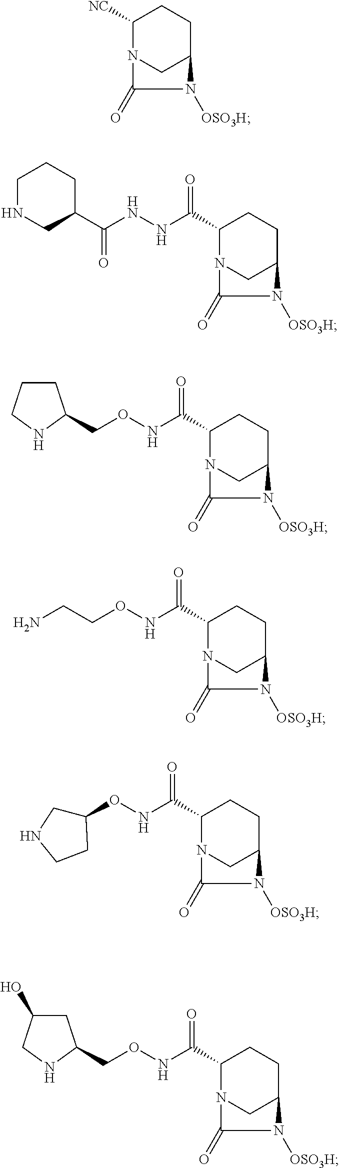 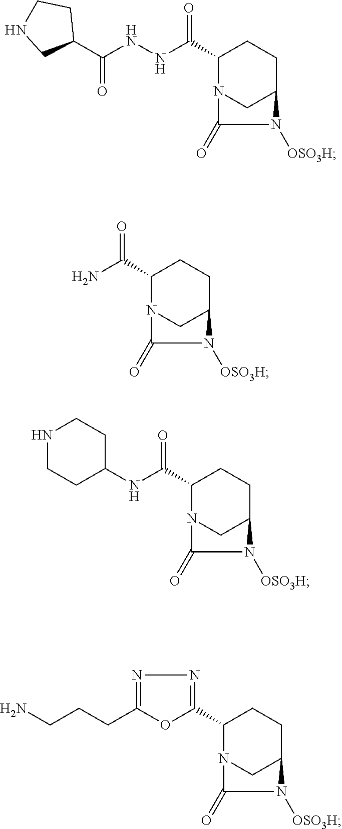
or a stereoisomer or a pharmaceutically acceptable salt thereof.

18. The method according to any one of the claims 14 to 17, wherein the beta-lactamase inhibitor or a pharmaceutically acceptable salt thereof is administered in an amount from about 0.01 gram to about 10 gram.

19. The method according to any one of the claims 14 to 17, wherein the compound of Formula (I) or a pharmaceutically acceptable derivative thereof is administered in an amount from about 0.01 gram to about 10 gram.

20. The method according to any one of the claims 14 to 17, wherein the beta-lactamase inhibitor, or a pharmaceutically acceptable salt thereof is administered in an amount from about 0.125 to about 4 gram per gram of the compound of Formula (I) or a pharmaceutically acceptable salt thereof.

21. The method according to any one of the claims 14 to 17, wherein the beta-lactamase inhibitor, or a pharmaceutically acceptable salt thereof, and the compound of Formula (I), or a pharmaceutically acceptable salt thereof, are administered in any of the following amounts:
(i) about 1 gram of the compound of Formula (I) or a pharmaceutically acceptable salt thereof, and about 0.25 gram of the beta-lactamase inhibitor, or a pharmaceutically acceptable salt thereof;
(ii) about 1 gram of the compound of Formula (I) or a pharmaceutically acceptable salt thereof, and about 0.50 gram of the beta-lactamase inhibitor, or a pharmaceutically acceptable salt thereof;
(iii) about 1 gram of the compound of Formula (I) or a pharmaceutically acceptable salt thereof, and about 1 gram of the beta-lactamase inhibitor, or a pharmaceutically acceptable salt thereof;
(iv) about 1 gram of the compound of Formula (I) or a pharmaceutically acceptable salt thereof, and about 2 gram of the beta-lactamase inhibitor, or a pharmaceutically acceptable salt thereof;
(v) about 2 gram of the compound of Formula (I) or a pharmaceutically acceptable salt thereof, and about 0.25 gram of the beta-lactamase inhibitor, or a pharmaceutically acceptable salt thereof;
(vi) about 2 gram of the compound of Formula (I) or a pharmaceutically acceptable salt thereof, and about 0.5 gram of the beta-lactamase inhibitor, or a pharmaceutically acceptable salt thereof;
(vii) about 2 gram of the compound of Formula (I) or a pharmaceutically acceptable salt thereof, and about 1 gram of the beta-lactamase inhibitor, or a pharmaceutically acceptable salt thereof;
(viii) about 2 gram of the compound of Formula (I) or a pharmaceutically acceptable salt thereof, and about 2 gram of the beta-lactamase inhibitor, or a pharmaceutically acceptable salt thereof;
(ix) about 0.5 gram of the compound of Formula (I) or a pharmaceutically acceptable salt thereof, and about 1 gram of the beta-lactamase inhibitor, or a pharmaceutically acceptable salt thereof; or
(x) about 0.5 gram of the compound of Formula (I) or a pharmaceutically acceptable salt thereof, and about 2 gram of the beta-lactamase inhibitor, or a pharmaceutically acceptable salt thereof.
